(19) United States
(12) Patent Application Publication
(10) Pub. No.: US 2009/0221082 A1
(43) Pub. Date: Sep. 3, 2009

(54) Microfluidic Evaporators And Determining Physical And/Or Chemical Properties Of Chemical Compounds Therewith

(75) Inventors: Mathieu Joanicot, Chatenay-Malabry (FR); Jacques Leng, Bordeaux (FR); Armand Ajdari, Paris (FR); Patrick Tabeling, L'Hayles-Roses (FR)

Correspondence Address: BUCHANAN, INGERSOLL & ROONEY PC, POST OFFICE BOX 1404, ALEXANDRIA, VA, 22313-1404, US

(73) Assignees: Rhodia Operations, Aubervilliers (FR); Centre National De La Recherché Scientifique, Paris Cedex 16 (FR)

(21) Appl. No.: 12/089,488

(22) PCT Filed: Oct. 5, 2006
(86) PCT No.: PCT/EP2006/009645

§ 371 Date: Oct. 8, 2008

Related U.S. Application Data
(60) Provisional application No. 60/724,127, filed on Oct. 6, 2005.

(51) Int. Cl. G01N 33/44; G01N 33/00; G01N 1/10; G01N 21/00

(52) U.S. Cl. 436/85; 422/68.1; 436/180; 436/164

(57) ABSTRACT

Microfluidic devices having a membrane allowing evaporation are useful for conducting a measurement or observation of compounds introduced therein.

### BRIEF SUMMARY OF THE INVENTION

The invention relates to a microfluidic device and to a process involving used of the device. The device comprises a membrane allowing evaporation. The process involves performing a measurement or observation of compounds introduced in the device.

Determination of the phase diagram of multicomponent systems is of importance in many realms: industrial formulation, protein cristallization, bottom up material assembly from spontaneous ordering of surfactant, polymeric or colloidal systems. Methods to reach this goal include thermal variations (in space or time) of samples of fixed concentrations or isothermal concentration by either removal of the solvent (osmosis, drying) external action on the solutes (sedimentation or dielectrophoresis for colloids), or studies of spontaneous interdiffusion in contact experiments. Depending on the application, one may want to access only the equilibrium phase diagram or gain additional information as to the metastable phases that can appear for kinetic reasons.

The invention introduces microfluidic tools for controlled isothermal concentration of a wide range of systems, covering solutions of ions, polymers, proteins, surfactants and colloidal suspensions.

The invention allows performing precise measures and/or sets of measures, and/or allows performing rapid measures and/or sets of measures, and/or allows performing simple measures and/or sets of measures, and/or performing measures and/or sets of measures with low amounts of matter.

### BRIEF DESCRIPTION OF THE DRAWINGS

FIG. 1 is a schematic representation, with a top view and side view, of a device and a processed composition of matter, according to an embodiment of the invention, wherein the device is multilayered and has a single blind extremity flowing channel, surrounded by a single closed evaporation chamber.

FIG. 2 is a schematic representation, with a top view and side view, of a device according to an embodiment of the invention, wherein the device is multilayered and has a 4 blind extremities flowing channels of different lengths, surrounded by a single closed evaporation chamber.

FIG. 3 is a schematic representation, with a top view and side view, of a device and a processed composition of matter, according to an embodiment of the invention, wherein the device is multilayered, and has a single flowing channel surrounded by a single closed evaporation chamber, the flowing channel having an accumulation chamber.

FIG. 4 is a schematic representation, with a top view and side view, of a device according to an embodiment of the invention, wherein the device is monolayered, and has 2 blind extremities flowing channels, surrounded by a single open-air evaporation zone.

FIG. 5 is a schematic representation, with a top view and side view, of a device according to an embodiment of the invention, wherein the device is monolayered, and has a single flowing channel surrounded by a single open-air evaporation zone, the flowing channel having an accumulation chamber.

FIG. 6 is a schematic representation, with a top view and side view, of a device according to an embodiment of the invention, wherein the device is monolayered, and has a two flowing channels surrounded by a single open-air evaporation zone, the flowing channels having each an accumulation chamber.

FIGS. 7 and 8 are schematic representations, with side views, of devices according to embodiments of the invention, wherein the device is multilayered and has a single flowing channel surrounded by a single closed evaporation chamber, the flowing channel having a tri-dimensional accumulation chamber.

FIG. 9 shows measures and/or computations thereof relating to fluorescent tracers in the examples

FIG. 10 shows measures and/or computations thereof relating to concentrations in the examples.

FIG. 11 shows photographs, measures and/or computations thereof relating to crystal growth in the examples.

FIG. 12 shows photographs relating to crystal growth in the examples.

### DETAILED DESCRIPTION OF THE INVENTION

### Device

The invention relates to a monolayered or multilayered device comprising:

at least one elongated flowing channel (1, 21a, 21b, 41a, 41b, 61a, 61b, 71, 81), for flowing a liquid therein, having a length and a width and/or depth, in a first layer, said channel having a closed section,

at least one evaporating chamber (2, 72, 82), for flowing a gas therein, in a second layer, said chamber having a closed section, or at least one evaporating open-air zone (42, 62),

the evaporating chamber or zone surrounding the width and/or depth of the channel, along at least ⅓ of the length, preferably at least ½, preferably at least ⅔, and/or along at least 10 times the width and/or depth of the flowing channel, preferably at least 100 times,

the flowing channel and the evaporation chamber or zone being separated by an evaporation membrane (3, 43, 73, 83).

The device is preferably a microfluidic device.

The length of the flowing channel(s) is preferably of at least 100 times its width and/or depth.

The closed cross section of the flowing channels can be for example comprised of 4 elongated walls defining width and depth in the section plan, and length in the elongation plan.

The device preferably comprises at least one microfabricated element (4a, 4b). The microfabricated element can be comprised of:

an elastomeric material, such as a silicone such as polydimethylorganosiloxane (PDMS), or

a non elastomeric material such as a metal or glass, or non elastomeric plastic.

Microfabrication techniques and materials are know by the one skilled in the art, especially in the field of microfluidics.

A closed evaporation chamber is typically provided with a multilayered device. An evaporating open air zone is typically provided with a monolayered device. Typically a multilayered device associates several layers of microfabricated elements. The association can be performed by bonding a microfabricated element onto the other, for example by pasting or welding. Typically a monolayered device has a single microfabricated element.

The evaporating chamber or zone surrounds the width and/or depth of the channel. By surrounding it is meant that the chamber or zone is close to the flowing channel(s) and that for at least one segment of the flowing channel(s), said flowing channel(s) and the evaporation chamber or zone are separated by an evaporation membrane or a part thereof. The surrounding can be also referred to a covering. However it is possible to have the evaporation chamber or zone on top of the flowing channel(s) as shown on FIG. 8, or to have the flowing channel(s) on top of the evaporation chamber or zone as shown on FIG. 9.

The evaporations chamber has preferably two channels allowing a gas inlet and a gas outlet.

For examples the device can comprise a first layer and an optional second layer, wherein:

the first layer comprises a first microfabricated element comprising the evaporation membrane and the flowing channel(s) carved into said first element, closed with a support plate (5) such as a glass or metal plate,

the optional second layer comprises a second microfabricated element comprising the evaporation chamber(s) carved into said second element, closed with the evaporation membrane of first layer.

The evaporation membrane is typically a part of a microfabricated element. It is preferred that the evaporation membrane be substantially not deflectable and is not deflected during the use of the device. Deflection can be controlled by managing the thickness of the membrane and/or its material. Deflection can be also controlled by managing the dimensions of the flowing channel(s) for example by using a channel(s) with a low with and/or depth is the segment surrounded by the evaporation chamber or zone. Deflection can be also controlled by managing the gas flow and/or the gas pressure. Upon flowing a gas deflection of the membrane should be avoided in order to prevent stopping a flow in the flowing channel(s).

The evaporation membrane typically closes a part of the section of the flowing channel(s) and of the evaporation chamber, and/or closes a part of the section of the flowing channel(s) and defines the evaporation zone.

It is preferred that the evaporation chamber(s) or zone(s) be elongated and substantially parallel to the flowing channel(s).

The flowing channel(s) can have a width and/or depth of less than 10 μm, preferably of less than 5 μm.

The flowing channel(s) can have a length of more than 1 mm, preferably of more then 5 mm, preferably of more than 10 mm.

The evaporation membrane can have a thickness of lower than 100 μm, preferably of lower than 50 μm.

The evaporation chamber(s) or zone(s) is preferably an elongated channel, open-air or closed, having a length and a width and/or depth, the width and/or depth being higher than the width and/or depth of the flowing channel(s).

In one embodiment the device comprises:

at least two flowing channels (21a, 21b or 41a, 41b, or 61a or 61b), being parallel on at least one segment thereof,

a single evaporation chamber (22) or zone (42, 62) surrounding the at least two flowing channels, or at least two evaporation chambers or zones, preferably evaporation channels, surrounding each at least one of the flowing channels.

In this embodiment the at least two flowing channels can have each a different length surrounded by the evaporation chamber(s) or zone(s).

The device can further comprise means for providing heat, such as means for heating a gas introduced into the evaporation chamber(s) or zone(s), said heat being optionally provided as a constant or as a gradient along at least a part of the channel(s). The device can thus comprise means for flowing a gas into the evaporation chamber or zone. For example the evaporation chamber can be connected to a gas dispenser such as a bottle with a vane, with a gas heater. The temperature of the gas is preferably controlled and/or measured.

The device typically comprises means (6) for providing a liquid into the flowing channel(s). Examples of such means include a reservoir or a syringe. The reservoir can be filled with any appropriate mean, such as a syringe, a bottle, a pipette, a capillary tube etc. . . . .

Typically the flowing channel(s) has:

an introduction extremity, linked to means (6) for providing a liquid into the flowing channel(s), and

an ending extremity, being linked to means for recovering matter comprised into the channels, or preferably being a blind extremity (7, 31, 51, 63a, 63b, 74, 84).

In one mode the flowing channel(s) does not extend beyond the evaporation chamber(s) or zone, has a blind extremity surrounded by the evaporation chamber(s).

In another mode the flowing channel(s) extends beyond the evaporation chamber(s) or zone (3), and has a blind extremity not surrounded by the evaporation chamber(s) or zone (s).

In one particular embodiment:

the flowing channel(s) extends beyond the evaporation chamber(s) or zone(s), and has a blind extremity not surrounded by the evaporation chamber(s) or zone(s), and

the flowing channel(s) have an enlarged blind extremity forming an accumulation chamber (31, 51, 63a, 63b, 74, 84), not surrounded by the evaporation chamber(s) or zone(s).

By “enlarged” it is meant the blind extremity has a width and/or depth substantially higher than the width and/or depth or the flowing channel(s), preferably of at least 5 times higher. In one mode the accumulation chamber is enlarged only for its depth or width. It can be referred to a 2-dimensionnal accumulation chamber (31, 51, 63a, 63b). In another mode the accumulation chamber is enlarged for its depth and its width. It can be referred to a 3-dimensionnal accumulation chamber (74, 84). 3-dimensionnal accumulation chambers can allow accumulating more composition of matter, and can allow having much more composition of matter there than in the flowing channel(s).

The accumulation chamber is usually not connected to means for removing matter therefrom. Once compositions of matter have accumulated there and once measures and/or observations have been performed, the device (or at least the part of the device comprising the flowing channel(s)) is usually disposed of. There is usually no connection from the accumulation chamber to out of the device, except via the flowing channel(s). On the contrary for example a reservoir is connected to the flowing channel and to out of the device (for example with an open-air opening as represented to the figures).

In one embodiment the flowing channel(s) has two introduction extremities, both being linked to means for providing a liquid into the each of the extremities of the flowing channel(s). Such means include reservoir, syringes etc. . . . .

Usually the device will comprise means for mixing at least one chemical compound and a carrier liquid to be at least partly evaporated along the channel(s), upstream the flowing channel. Conventional pots and/or mixers can be used. In one embodiment the device can be associated to a microfluidic mixing device allowing providing a range of concentrations of mixture along time, and/or topography, for example comprising several mixing output channels, connected to several flowing channels, for example a Whitesides-like Microfluidic mixing device.

According to a specific embodiment the device has:

at least two flowing channels, and

at least one open-air evaporation zone, or

at least two flowing channels, and

at least one closed evaporation chamber.

According to a specific embodiment the device has:

at least one, preferably at least two, accumulation chamber(s) which is not surrounded by an evaporation membrane, and

at least one open-air evaporation zone, or

at least one, preferably at least two, accumulation chamber which is not surrounded by an evaporation membrane, and

at least one closed evaporation chamber.

### Process for Determining Properties—Process of Use of the Device—Applications

The invention also relates to a process for determining physical and/or chemical properties of chemical compounds or mixtures of chemical compounds, comprising the steps of:

a) providing:
a liquid mixture of a carrier fluid and one or several candidate chemical compound(s) into the flowing channel(s) of the device, preferably microfluidic device, and
a gas flow in the evaporation chamber, or an open-air contact to the evaporation zone, or a gas flow surrounding the evaporation zone,
   b) flowing the liquid mixture along at least a part the flowing channel(s), and removing at least partly the carrier fluid from the channel by evaporating through the membrane into the evaporation chamber or into the open-air evaporation zone, thereby providing in the flowing channel(s) solid or liquid compositions of matter with different concentrations of carrier and residence times along the channel(s) (for example variations upon time and/or space along the flowing channels(s)), and thereby optionally providing accumulation of compositions of matter in the accumulation chamber if the device has such a chamber,
   c) performing at least one measurement or observation of a composition of matter:
in the channel(s), at least one point along the channel(s), and/or
in the accumulation chamber.

Typically the flowing in step b) can be induced by removal of the carrier fluid, the flowing being optionally stopped upon solidification of the mixture (dynamic movement of carrier fluid). Without being bound to any theory, it is believed that motion (flow) of the liquid in the flowing channel(s) is induced by evaporation of the carrier. The liquid can flow to an extremity of the flowing channel(s), optionally to an accumulation chamber. Alternatively the liquid can flow in a part the flowing channel(s), to a point where is solidifies completely, beyond which there is no flowing. Complete solidification can be due to a lack of a sufficient amount of carrier further to evaporation. Preferably substantially no movement of fluid is induced in the accumulation chamber, if the device has such a chamber, and the concentration of carrier fluid in the chamber being of zero or higher.

It is preferred that the carrier fluid be chemically compatible with the channel(s) material(s) and the evaporation membrane material(s), and such that the membrane/carrier system allow transfer of the carrier trough the membrane (for example by having appropriate membrane thickness, porosity and/or material). Compatibility means that the carrier fluid should not chemically degrade to flowing channel(s)'s and membrane's material, and should not be repelled by the membrane's material.

The liquid mixture comprises a carrier and one or several candidate chemical compound(s). In the embodiment where the liquid mixture comprises several candidate compounds, these several compounds can represent a mixture and/or association and/or reagents, that is to be studied and/or screened via the measurement and/or observation.

The gas flow is provided by appropriate means. The gas flow is at a temperature and/or at a flow rate allowing evaporation of the carrier and removal thereof from the evaporation chamber or evaporation zone. Open air contact might provide enough heat and movement to do so.

In step a) a liquid is provided in the flowing channel(s). The liquid is typically by appropriate means, for example by a reservoir. The liquid is thus a placed at an inlet(s) of the flowing channel(s).

The candidate chemical compound(s) can form a solid along the channel(s), said solid being:
dispersed in the carrier fluid (the solid can be a precipitate, or a crystal for example), optionally accumulating in a part of the flowing channel(s) and/or in the accumulation chamber if the device has such a chamber, or
completely solidified.
   The solid can form because of removal of the carrier by the evaporation.

In one embodiment the compositions of matter are varied, in space along the channel(s) and/or in time at one point along the channel(s), by varying the gas flow characteristics, such as stopping and/or re-starting the gas flow or varying the speed flow, or varying the temperature. It is believed that varying the gas flow characteristics, controls distribution of solids and/or concentrated phases in the carrier fluid, allows for example relaxation and/or diffusion of solids back along the flowing channel(s) upon lowering or stopping evaporation flow (lowering gas flow and/or stopping gas flow and/or lowering temperature).

In one embodiment:

the device has an accumulation chamber,

the candidate compound(s) forms a solid and/or a concentrated phase along the channel(s) that accumulates in the accumulation chamber, and

a flow of solids in time and/or in space is controlled, as diffusion of solids back in the channel is controlled.

In one embodiment the composition of matter in the accumulation chamber varies along time, for example the concentration varies (usually increasing by accumulation).

It is believed that the accumulation chamber can help in addressing diffusion artifacts when several candidate compounds are present in the liquid. One would typically perform measurements and/or observations at the accumulation chamber, and optionally at the border thereof, in the flowing channel(s) just before the accumulation chamber. The amount of a solute candidate compound in the accumulation chamber vis a vis the amount in the flowing channel can be determined. If two solute candidate compounds are introduced in a known ratio in the flowing channel, then the same ratio will be retrieved in the accumulation chamber, without possible diffusion artifacts that can be observed in the flowing channel. The accumulation chamber can thus improve precisions of measurements, observations and/or datas obtained from computations therefrom.

In one embodiment one can perform a first measure in the accumulation chamber at time t1 with a proportion of carrier fluid of c1, and then perform at least one second measure in the accumulation chamber at time t2 with a proportion of carrier fluid of c2 being lower than c1 (as matter accumulates). One would then compute the measures to determinate a useful property or parameter.

In one embodiment one can introduce first in the flowing channel a mixture of candidate compounds A and B with relative proportion of respectively a/(a+b) and b/(a+b) then one can perform a measure in the accumulation chamber for said proportions, then one can introduce a second proportion of a′/(a′+b′) and b′/(a′+b′), and then perform a measure in the accumulation chamber for proportions respectively of (a+a′)/(a+a′+b+b′) and (a+a′)/(a+a′+b+b′). One would then compute the measures to determinate a useful property or parameter.

In one embodiment the device comprises at least two flowing channels (21a, 21b), being parallel on at least one segment thereof. In one aspect of this embodiment the device can have accumulation chambers associated with the flowing channels, and one can perform the following:

first step:
one introduces in a first flowing channel a mixture of candidate compounds A and B with relative proportion of respectively a/(a+b) and b/(a+b) then one performs a measure in the in the accumulation chamber of said channel for said proportions,
one introduces in a second flowing channel a mixture of candidate compounds A and B with relative proportion of respectively a′/(a′+b′) and b′/(a′+b′) then one performs a measure in the in the accumulation chamber of said channel for said proportions,

optionally, then second step:
one reiterates the measure with same proportions in respective accumulation chambers but with different concentrations of carrier fluid.

In another aspect of the embodiment one liquid mixture being provided into one of the flowing channels is a reference liquid mixture, having a known physical and/or chemical property. One would typically measures a property for one or several channel(s) having a candidate compound and compare it to a measure performed for the channel having the reference liquid mixture. One would compute to measures and/or comparison to determine a useful property or parameter.

In still another aspect of the embodiment:

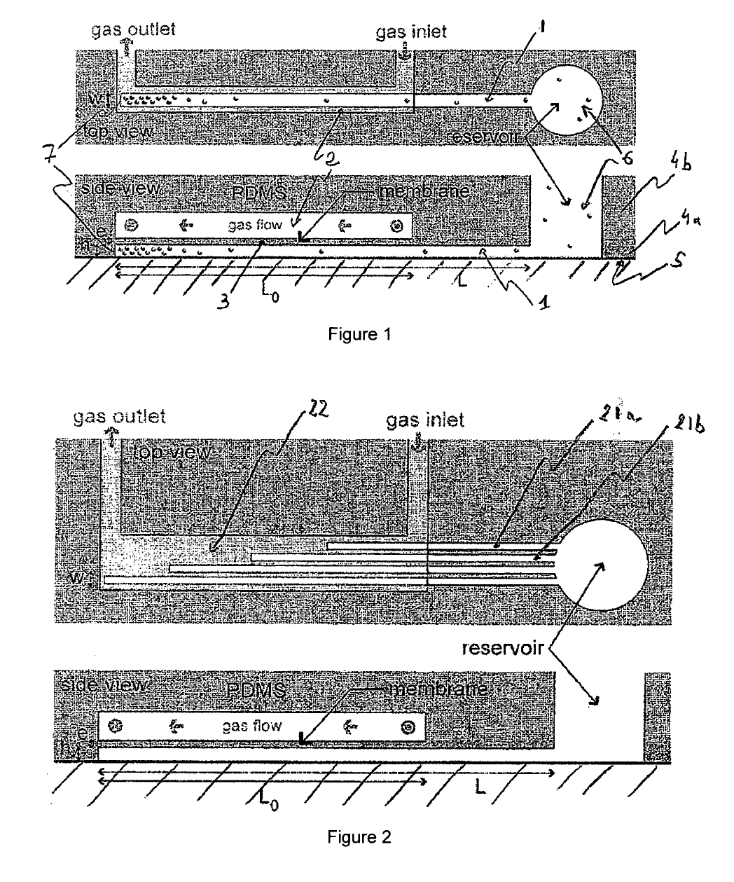

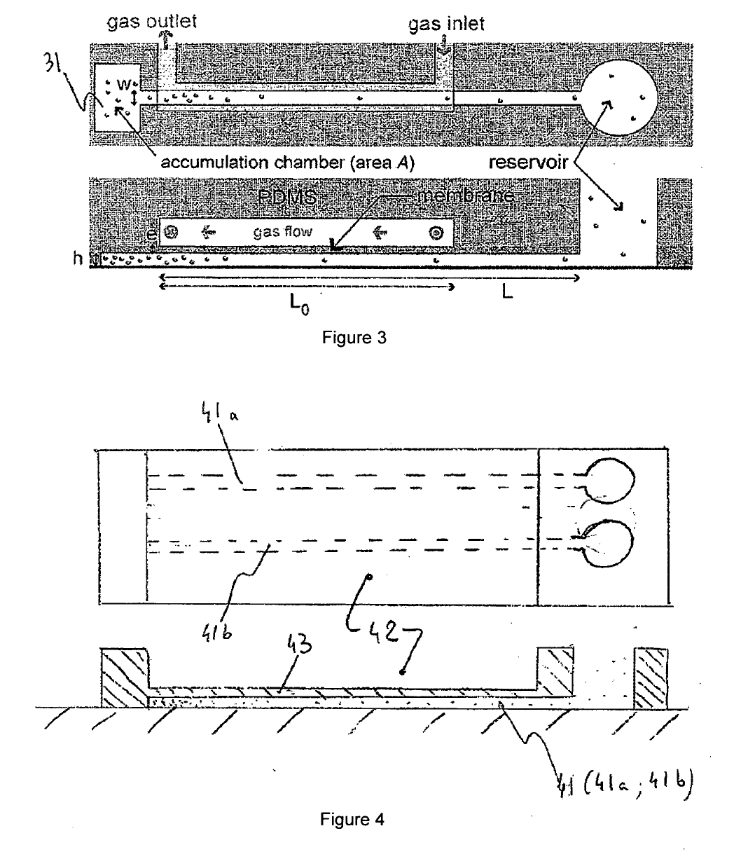

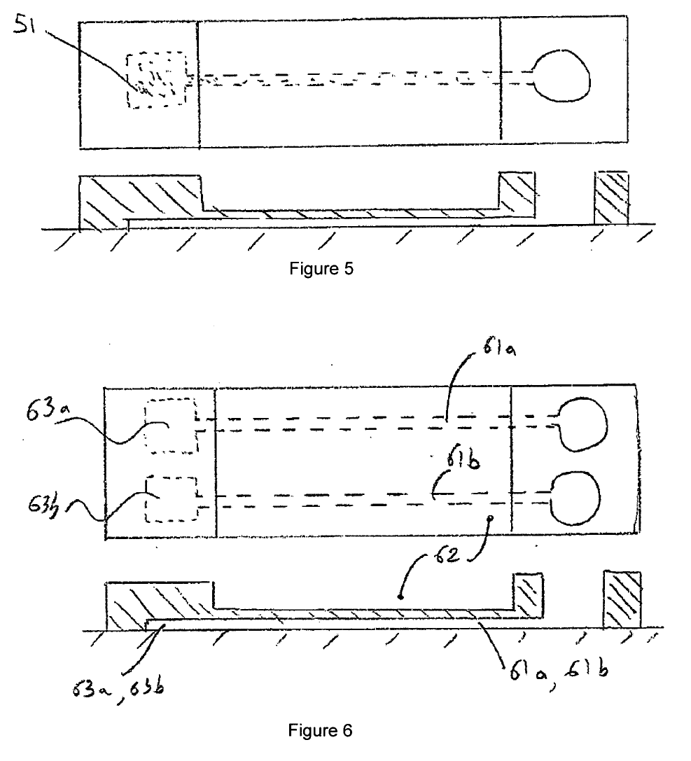

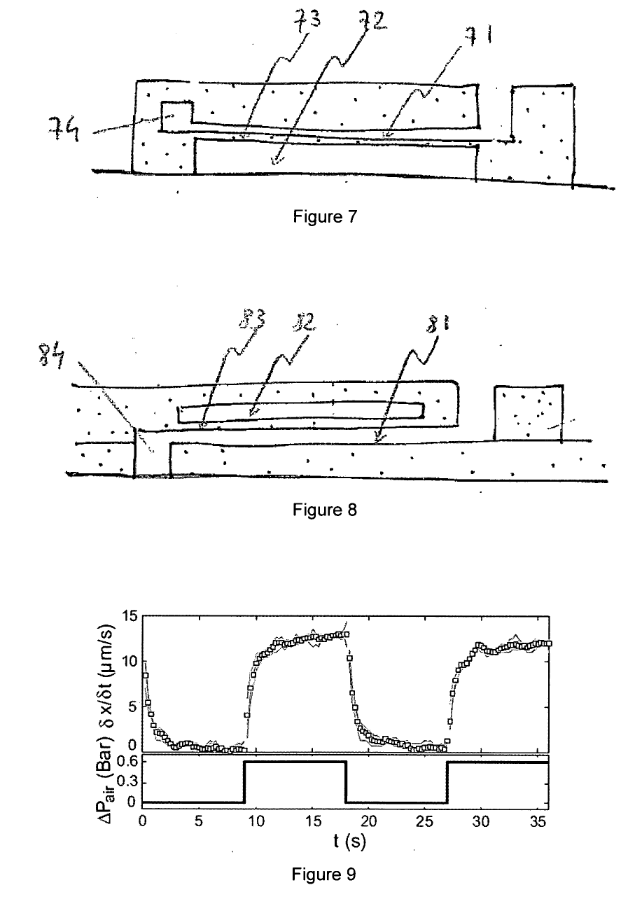

the at least two flowing channels have each a different length surrounded by the evaporation chamber(s) (the evaporation can have a constant length with flowing channels having different lengths as shown on FIG. 2, or the flowing channels can have the same length with the evaporation chamber(s) being such that it surround different lengths of the flowing channel(s)),

the same liquid mixture is introduced in at least the two channels,

concentration in carrier fluid has a different rhythm in the different channels, and

a solid and/or a concentrated phase forms at different rhythms in the different channels.

The rhythms refer to formation of a solid or concentrated phase as of time and/or space along the channels. The different rhythms can be used to generate information about solubility and/or crystallization kinetics and/or crystal growth kinetics.

In still another aspect of the embodiment two different liquid mixtures (for example as of chemical compositions and/or concentrations) of an identical or different carrier fluid and one or several (for example mixture and/or association and/or reagents) identical or different candidate chemical compound(s) are flowed into the flowing channels. The different liquid mixtures, can be provided with an associated microfluidic mixing device. One can thus generate information about the different mixture and candidate compounds and associations/reactions/interactions thereof or therewith. In one particular fashion:

the carrier fluid is identical in each channel,

the candidate chemical compound(s) are different in each channel and/or are mixtures of identical compounds with different concentrations in each channel, and

the process provides an array of:
different concentrations of carrier fluid and or residence times along the channels, with
different compositions of matter through the parallel segments of the channels.

In another particular fashion:

the carrier fluid is different in each channel,

the candidate chemical compound(s) is identical in each channel and/or are mixtures of an identical compound with another compound, optionally with different concentrations, and

the process provides an array of:
different concentrations of carrier fluid and or residence times along the channels, with
different compositions of matter through the parallel segments of the channels.
   The array can constitute libraries of compositions of matters. One can perform measurements or observations for all or a part or the compositions of matter of the array (thus of the library).

The measurements and/or observations can be performed by any appropriate means, methods, and techniques. These include conventional techniques used in chemistry, physics and physico-chemistry, including those more recently developed for microfluidics. For example one can implement spectroscopy (for example Raman, Infra-red, UV), fluorescence, conductimetry, rheology measures (for example with using magnetic particles) and/or optical observations (usually with a microscope, optionally using a polarizer), preferably by image analysis. The measurements and/or observations can be are computed, optionally with using all or a part of the process parameter (flow rates, concentrations, natures of compounds) to provide:

phase diagrams, such a binary, tertiary or further phase diagrams,

crystallization diagrams,

crystallization kinetics datas,

crystal growth kinetics datas,

solubility datas,

nucleation of solids datas,

kinetics of chemical reactions,

formulation information, for example in the field of pharmaceuticals, cosmetic compositions, detergent compositions, coating compositions, and/or

material engineering datas, for example for engineering inorganic compounds, or for engineering polymeric compounds.

The candidate chemical compound(s) can comprise:

biological molecules, for example biological polymers, and/or

non biological molecules, for examples synthetic polymers, surfactants, inorganic particles.

In some embodiments the carrier fluid is a solvent or at least some of candidate chemical compounds, the mixture of the carrier fluid being and the candidate being in a form of:
a solution, or
an emulsion of liquid droplets, or
a dispersion of solid particles.

The invention also relates to a process of screening candidate compounds, comprising a process described above, and/or comprising the step of introducing the candidate compounds in the device above. One would typically performs measurements and/or observations one several compounds or mixtures of compounds and then identified a useful compounds or mixture of compounds for an optimum property.

Further details, embodiments, and/or advantages of the invention appear on the following examples which are not limitative.

### EXAMPLES

It is found that in standard microsystems built of PolyDiMethylSiloxane (PDMS), spontaneous water permeation through the PDMS matrix induces flows that can be used to concentrate colloids. We have engineered specialized microgeometries that allow us to control spatially and temporally the evaporation process as well as the resulting concentration of solutes.

After a brief description of the micro-devices, we demonstrate first our control of the concentration process on dilute aqueous solutions of fluorescein and nanoparticles. We then report controlled nucleation and growth of crystals of potassium chloride (KCl) in micro-channels, and extract various thermodynamic quantities (solubility, crystal density) as well as kinetic features (sensitive to the rate of concentration). After a discussion of the large spectrum of experiments that can be performed in similar microfabricated devices, we conclude on the versatility and large applicability to soft matter systems.

### The Devices

The devices used are two-layer PDMS on glass Microsystems (FIG. 1). The microchannels (flowing channel(s)) of the bottom layer are filled with the solution of interest, while air is circulated through the microchannels (evaporation chamber) of the top layer so as to remove the water that pervaporates through the thin membrane of PDMS that separate the two networks where they overlap (thickness e in the 10-30 μm range). Many combinations of geometries for the bottom and top networks can be envisaged. We focus here on the simple “finger” geometry of FIG. 1, a dead end (blind extremity) channel of rectangular cross section (height h, width w, length L) connected to a larger (millimetric) feeding reservoir containing the solution to be concentrated. A terminal section of length L₀<L (typically mms to cms) is covered by the water removal network. The operation principle is simple: water in the bottom channel pervaporates through the thin membrane, which induces a compensating flow from the reservoir and concentration of solutes at the finger tip. This is similar to concentration at the boundary of a drying droplet, without the motion and shear of the concentration zone due to the recess of the liquid-air interface, and without spurious convective flows thanks to the confinement. In addition, many microchannels of various geometries and types can be fabricated on a single chip, so that we can run many experiments in parallel, from a single reservoir or multiple ones. The small dimensions lead to fast thermal regulation that permits isothermal studies, and we can directly observe the induced phases and phenomena thanks to the PDMS transparency.

General processes for preparing multilayer microfluidic devices are for example described in document WO 01/01025, which is incorporated by reference. These processes can be adapted simply to obtain the geometry (topography, design, length, width, depth, etc. . . . ) of the present invention.

FIG. 1: Sketch of the finger geometry: top and side views showing the gas and liquid layers and the thin PDMS membrane in between (typical dimensions: e=10 μm, h=20 μm, w=200 μm, L₀=10 mm).

### Control of Flow and Particle Concentration

To quantify the induced flow, we adapt the analysis, anticipating that evaporation occurs here mostly through the thin membrane, at a volumic flow rate of water vₑ (it has dimension of a velocity). Mass conservation then sets the height-averaged velocity in the microchannel: v(x)=−vₑ(x/h). Its amplitude rises with the distance x from the dead-end (x=0) up to v₀=vₑ(L₀/h) at the end of the evaporation zone (x=L₀). v(x)=−v₀ in the evaporation-free section of the finger L₀≦x≦L.

With our dedicated systems we induce large values of vₑ (in the 50 nm/s range) due to the thin membranes (permeation yields a limit scaling as 1/e) and to the dry air flown through the top network (velocities of order cm/s) that reduces the diffusive boundary layer. More importantly, we gain a spatial and temporal control on vₑ by designing the geometry (evaporation is negligible but for chosen locations) and by tuning in time the air flow and thus vₑ. Quantitative temporal control of the flow field is clear from the motion of tracers at a given location as the air flow is successively turned on and off (FIG. 9).

FIG. 9: Velocity of fluorescent tracers at a fixed location in a finger in response to the switching on and off of air circulation in the water removal network (the instantaneous velocity (gray lines) is obtained from individual trajectories of 1.1 μm diameter tracer gathered by Particle Tracking Velocimetry. The symbols are for the mean velocity averaged on ≈10 trajectories. L₀=12.5 mm, h=22 μm, e≈20 μm.).

When on, tracer velocities of order 13 μm/s are observed, corresponding to Tₑ=h/vₑ≈10³ s and vₑ≈22 nm/s. The velocity drops below 1 μm/s when the air flow is turned off, after a response time of a few seconds, compatible with that of the water flux through the thin PDMS layer, e²/D_{PDMS}˜0.5 s for e≈20 μm and a diffusion coefficient for water in PDMS D_{PDMS}˜10⁻⁹ m²/s.

Control of the flow field translates into that of the induced concentration process. For the simplest case of a dilute species of diffusion coefficient D in a finger, in a one-dimensional description the conservation equation dₜc+dₓJ=0 relates the concentration c(x,t) and flux J(x,t)=cv−Ddₓc. We focus now on steady evaporation and thus constant v(x) in time, with a reservoir at fixed concentration c₀. The physics at work is simple: the flow convects the solute towards the dead end where it accumulates. The current of solute injected into the finger is steady J₀=c₀v₀. Backwards thermal diffusion against the flow controls the width of the accumulation zone, which is p=(Dh/vₑ)^{1/2}=(DTₑ)^{1/2}<L₀ for strong flows or long fingers v₀L₀/D>>1. At distances larger than p, diffusion is negligible and the suspension is simply concentrated by water removal at constant particle flux c(x)v(x)=c₀v₀. Altogether, after a transient of duration p²/D=Tₑ, the profile is well approximated by a Gaussian (because of the linearity of v(x)) increasing linearly in time, “fed” by a steady hyperbolic ramp delivering a current J₀=c₀v₀:

c(x,t)=c₀v₀t(2/p²π)^{1/2} exp(−x²/2p²)+c₀R(x) (1)

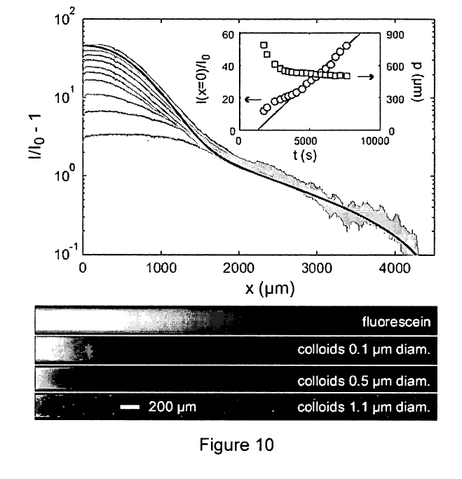

with R(x)≈L₀/x for p<<x<<L₀. Experiments on solutions of fluorescein and nanocolloids quantitatively support this analysis (FIG. 10).

FIG. 10: Evaporation-induced concentration. Top: log-linear plot of fluorescence intensity against position at different times for a finger filled with a aqueous solution of fluorescein. The fit corresponds to the prediction (1), with a Gaussian hump of width p # 500 μm, that increases linearly in time (insert), yielding vₑ=15±2 nm/s and Tₑ=950±100 s. (c₀=5 10⁻⁵ M, L₀=4.7 mm, h=15 μm, e # 25 μm). Bottom: images from four fingers in similar conditions showing that the size of the accumulation zone varies with the size (and diffusion coefficient) of the markers.

Concentration at the tip increases as dc(x=0)/dt=(2/π)^{1/2}(ve/h)^{3/2}c₀L₀D^{-1/2}, offering many means of kinetic control of the process: through geometrical features L₀, h and e (that affects vₑ), and through operational parameters c₀ and vₑ. The latter can be modulated during an experiment, which allows us to pinch or relax concentration profiles, at a diffusion-limited response time ˜p²/D=h/vₑ=Tₑ (independent of the species), much larger than the response time of the flow (minutes instead of seconds).

### Controlled Crystal Nucleation and Growth

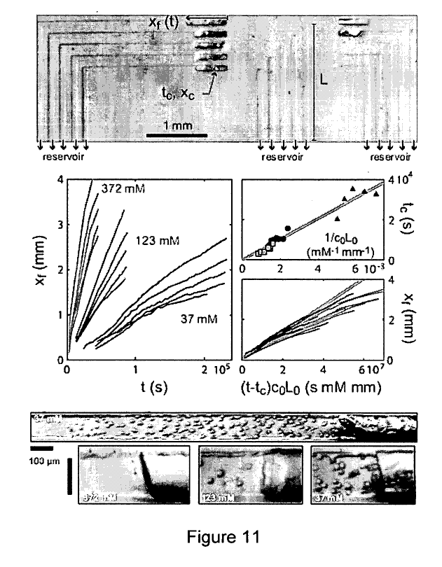

This control permits the study of phase transitions as we now show on a well characterized system, KCl aqueous solutions. We use a microchip with multiple “fingers” of different lengths L₀ originating from the same reservoir, and perform a few experiments, at the same steady air flux, with different initial concentrations. Upon concentration, we observe in each finger the nucleation of crystals close to tip, at x_{c} and t_{c} (FIG. 11), and then their subsequent growth with a front location at x_{f}(t). The time scales involved vary widely with initial concentration c₀ and finger length L₀. However, we can rescale data for both nucleation and growth by accounting for the concentration rate proportional to c₀L₀.

Nucleation occurs at t_{c}˜K (c₀L₀)⁻¹, which from (1) suggests a nucleation concentration c_{c}=K√(2/π).D^{-1/2} Tₑ^{-3/2}. For KCl (c_{c}=4.0±0.5 M, D # 2.0±0.2 10⁻⁵ cm²/s at this temperature), this is consistent with the experimental value of K # 5.5±1 sMm and a reasonable value for Tₑ=h/vₑ=800±50 s, p # 1200 μm. The good resealing of crystal growth data, suggest that it is limited by the solute feed at J₀=c₀v₀=c₀L₀/Tₑ. Further, if the growing crystal at solute concentration cₓ fills the channel, the initial growth rate should follow from mass conservation [(dxₜ)/dt]=J₀/cₓ=(c₀L₀)/(Tₑ c_{X}). The observed value (c₀L₀)⁻¹[(dxₜ)/dₜ]˜0.6±1.10⁻⁴ sMm, yields a value cₓ=20±2 M in reasonable agreement with Handbook value (26 M). The decrease of the growth rate in time for each channel likely follows from that of the evaporation length as the crystal grows.

This demonstrates a possible use of our devices. Feeding a few fingers with a system of reference (e.g. KCl or fluorescent probes) provides a local calibration of the value of Tₑ (to account for variations from device to device). Then measurements of t_{c} and front motion in other fingers fed with the solution to analyze yield estimates of c_{c}D^{1/2} and of the solute concentration cₓ in the phase that nucleates.

Further, with our microdevices we can investigate the influence of the kinetics of the concentration process on the morphologies and phases produced. We indeed observe on KCl solutions that both the nucleation scenario and the crystal growth process vary when the initial concentration c₀ and thus the concentration rate are changed. In the studies reported above, we reproducibly observe demixing of the solution into what looks like droplets in front of the growing crystal, but only at the lowest initial concentration examined (FIG. 11 bottom). Remarkably, the various transient organizations (nucleation scenario, presence or absence of droplets) do not affect the rescalings of FIG. 11, possibly due to the narrow metastable region of KCl and the robustness of mass conservation arguments.

FIG. 11: Top: view of the chip with 15 microchannels with different evaporation lengths L₀ (the bottom of the frame is the end of the evaporation zone). In each channel, the evaporation-induced concentration leads to crystallization of KCl solutions at x_{c} after a time t_{c}. Crystal growth is then monitored x_{f}(t). Middle: Left: crystal fronts x_{f}(t) for various evaporation lengths L₀ and initial concentrations c₀=37 mM (bottom), c₀=123 mM (middle), c₀=372 mM (top). Right: plots of nucleation time t_{c} against 1/c₀L₀, and front growth x_{f} against c₀L₀( t̅t̅_{c}). Bottom: Crystals growing at various c₀: “droplets” in front of the crystal are only visible at low concentrations.

Benefits from Microfabrication

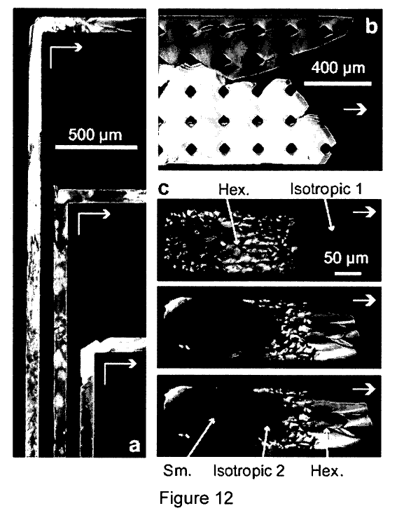

We have shown how microfabrication provides us with control, through flexibility in the geometrical parameters (e, h, L₀, L), and by permitting time regulation. In addition, the following can be done:
We can parallelize tests so as to perform rapid screening with minute amount of material. We have only touched upon the corresponding potentialities with our 15 finger chip (FIG. 4). In a more elaborate system, a distribution unit will feed many reservoirs with different solutions.
Geometries are not limited to fingers, opening up possibilities. Let us illustrate this on an important example. The analysis of ill-characterized mixtures faces in the finger geometry a problem common to many other methods (drying, field induced concentration, . . . ): different solutes are concentrated at different rates (p depends on D) and their proportions in the accumulation zone differ (in an a priori unknown way) from those in the reservoir. Geometrical design provides at least two solutions. One is to fabricate very long serpentine fingers and focus on the hyperbolic concentration ramp (R(x) in equation (1)) where all species are concentrated alike. A second strategy consists in adding a chamber of area A (thickness h) at the end of the finger with pervaporation limited to the finger. The accumulation area _{?}A+pw is essentially independent of p (or D) as long as p<<A/w (with A a few mm² and w a few hundred microns, this encompasses anything from ions to micron-sized colloids), restoring homotetic concentration from the reservoir.
We can force the growing phases through turns using wiggly fingers, or through arrays of obstacles of controlled shapes embedded in chambers (FIG. 12, a and b). This provide insights into growth mechanisms and the role of epitaxy as one can force various angles between the growth direction and the crystalline axis of the growing phase.
The PDMS devices presented here will work only with a limited set of solvents that do not swell this elastomer. However microfabrication permits the extension of the concepts presented here to other materials. The bottom layer of channels can for example be etched in an impermeable solid matrix (e.g.; glass for the “floor” and “side-walls”) separated from the air-circulating network by a thin membrane of a chosen porous materials. Similar sandwich micro-constructions have been used in other contexts to permit exchange between two layers of solvent carrying networks.

Beyond ionic solutions and colloids, the invention can be used on on surfactant solutions and on biomaterial crystallization. Experiments on Sodium Dodecyl Sulfate (SDS), allows to observe sequences of many different mesophases in a given microchannel (FIG. 12, c), and more importantly complex and intriguing dependences of that sequence and of the texture of each phase on the kinetics and history of the concentration process. At the same time, data for nucleation and front growth obey rescalings akin to those of KCl, showing our kinetic control.

FIG. 12: (a, b): Birefringent Cu₂SO₄ crystals grown through channels with sharp turns and through a chamber with diamond-section pillars. (c): evolution in time of the phase and texture pattern for a SDS solution as observed through crossed-polarizers (top to bottom, t=45, 60, 70 min, c # 40 mM; Isotropic 1=micellar solution, Hex.=hexagonal phase, Isotropic 2=cubic or re-entrant micellar solution, Sm.=Smectic phase). The arrows indicate the growth direction.

1.-50. (canceled)

51. A microfluidic monolayered or multilayered device comprising:
at least one elongated flowing channel, for flowing a liquid therein, having a length and a width and/or depth, in a first layer, said channel having a closed section,
at least one evaporating chamber, for flowing a gas therein, in a second layer, said chamber having a closed section, or at least one evaporating open-air zone,
the evaporating chamber or zone surrounding the width and/or depth of the channel, along at least ⅓ of the length and/or along at least 10 times the width and/or depth of the flowing channel,
the flowing channel and the evaporation chamber or zone being separated by an evaporation membrane.

52. The microfluidic device as defined by claim 51, wherein the evaporation chamber(s) or zone(s) is/are elongated and substantially parallel to the flowing channel(s).

53. The microfluidic device as defined by claim 51, wherein the flowing channel(s) has/have a width and/or depth of less than 10 μm.

54. The microfluidic device as defined by claim 53, wherein the flowing channel(s) has/have a length of more than 1 mm.

55. The microfluidic device as defined by claim 51, wherein the evaporation membrane is substantially not deflectable.

56. The microfluidic device as defined by claim 51, wherein said device comprises at least one microfabricated element.

57. The microfluidic device as defined by claim 56, wherein said at least one microfabricated element is comprised of:
an elastomeric material, or
a non-elastomeric material.

58. The microfluidic device as defined by claim 51, wherein:
a first layer comprises a first microfabricated element comprising the evaporation membrane and the flowing channel(s) carved into said first element, closed with a support plate,
an optional second layer comprises a second microfabricated element comprising the evaporation chamber(s) carved into said second element, closed with the evaporation membrane of the first layer.

59. The microfluidic device as defined by claim 51, wherein the evaporation membrane has a thickness of lower than 100 μm.

60. The microfluidic device as defined by claim 59, wherein the evaporation membrane:
closes a part of the section of the flowing channel(s) and of the evaporation chamber, and/or
closes a part of the section of the flowing channel(s) and defines the evaporation zone.

61. The microfluidic device as defined by claim 51, wherein the evaporation chamber(s) or zone(s) is/are an elongated channel, open-air or closed, having a length and a width and/or depth, the width and/or depth being higher than the width and/or depth of the flowing channel(s).

62. The microfluidic device as defined by claim 51, comprising:
at least two flowing channels, being parallel on at least one segment thereof, and
a single evaporation chamber or zone surrounding at least two flowing channels, or at least two evaporation chambers or zones, surrounding at least one of the flowing channels.

63. The microfluidic device as defined by claim 62, wherein the at least two flowing channels have each a different length surrounded by the evaporation chamber(s) or zone(s).

64. The microfluidic device as defined by claim 51, further comprising means for providing heat, said heat being optionally provided as a constant or as a gradient along at least a part of the channel(s).

65. The microfluidic device as defined by claim 51, comprising means for providing a liquid into the flowing channel(s).

66. The microfluidic device as defined by claim 51, comprising means for flowing a gas into the evaporation chamber or zone.

67. The microfluidic device as defined by claim 51, wherein the flowing channel(s) has:
an introduction extremity, linked to means for providing a liquid into the flowing channel(s), and
an ending extremity, being linked to means for recovering matter contained in the channel(s), or being a blind extremity.

68. The microfluidic device as defined by claim 51, wherein:
the flowing channel(s) does not extend beyond the evaporation chamber(s) or zone, has a blind extremity surrounded by the evaporation chamber(s), or
the flowing channel(s) extends beyond the evaporation chamber(s) or zone, and has a blind extremity not surrounded by the evaporation chamber(s) or zone(s).

69. The microfluidic device as defined by claim 51, wherein:
the flowing channel(s) extends beyond the evaporation chamber(s) or zone(s), and has/have a blind extremity not surrounded by the evaporation chamber(s) or zone(s), and
the flowing channel(s) has/have an enlarged blind extremity forming an accumulation chamber, not surrounded by the evaporation chamber(s) or zone(s).

70. The microfluidic device as defined by claim 51, wherein the flowing channel(s) has/have two introduction extremities, both being linked to means for providing a liquid into the each of the extremities of the flowing channel(s).

71. The microfluidic device as defined by claim 51, comprising means for mixing at least one chemical compound and a carrier liquid to be at least partly evaporated along the channel(s).

72. The microfluidic device as defined by claim 51, in combination with a microfluidic mixing device useful for providing a range of concentrations of mixture over time.

73. The microfluidic device as defined by claim 51, having:
at least two flowing channels, and
at least one open-air evaporation zone, or
at least two flowing channels, and
at least one closed evaporation chamber.

74. The microfluidic device as defined by claim 51, having:
at least one accumulation chamber which is not surrounded by an evaporation membrane, and
at least one open-air evaporation zone, or
at least one accumulation chamber which is not surrounded by an evaporation membrane, and
at least one closed evaporation chamber.

75. A process for determining physical and/or chemical properties of a chemical compound or a mixture of chemical compounds, comprising the steps of:
a) providing
a liquid mixture of a carrier fluid and one or several candidate chemical compound(s) into the flowing channel(s) of the device as defined by claim 51, and
a gas flow in the evaporation chamber, or open-air contact to the evaporation zone, or a gas flow surrounding the evaporation zone,
b) flowing the liquid mixture along at least a part the flowing channel(s), and removing at least partly the carrier fluid from the channel by evaporating through the membrane into the evaporation chamber or into the open-air evaporation zone, thereby providing in the flowing channel(s) solid or liquid compositions of matter with different concentrations of carrier and residence times along the channel(s), and thereby optionally providing accumulation of compositions of matter in an accumulation chamber, if the device has such a chamber, and
c) performing at least one measurement or observation of a composition of matter
in the channel(s), at least one point along the channel(s), and/or
in the accumulation chamber.

76. The process as defined by claim 75, wherein the carrier fluid is chemically compatible with the channel(s) material(s) and the evaporation membrane material(s), and is such that the membrane/carrier system allows transfer of the carrier fluid through the membrane.

77. The process as defined by claim 75, wherein flowing in step b) is induced by removal of the carrier fluid, the flowing being optionally stopped upon solidification of the mixture.

78. The process as defined by claim 75, wherein substantially no movement of fluid is induced in the accumulation chamber, if the device has such a chamber, concentration of carrier fluid in the chamber being zero or higher.

79. The process as defined by claim 75, wherein the candidate chemical compound(s) forms a solid along the channel(s), said solid being:
dispersed in the carrier fluid, optionally accumulating in a part of the flowing channel(s) and/or in the accumulation chamber if the device has such a chamber, or
completely solidified.

80. The process as defined by claim 75, wherein the compositions of matter are varied, in space along the channel(s) and/or in time at one point along the channel(s), by varying the gas flow characteristics, by stopping and/or re-starting the gas flow or varying the speed flow, or varying the temperature.

81. The process as defined by claim 75, wherein varying the gas flow characteristics, controls distribution of solids and/or concentrated phases in the carrier fluid, allows for relaxation and/or diffusion of solids back along the flowing channel(s) upon lowering or stopping evaporation flow.

82. The process as defined by claim 75, wherein:
the device has an accumulation chamber,
the candidate compound(s) forms a solid and/or a concentrated phase along the channel(s), that accumulate in an accumulation chamber, and
a flow of solids in time and/or in space is controlled, as diffusion of solids back in the channel is controlled.

83. The process as defined by claim 75, wherein compositions of matter in an accumulation chamber varies along time.

84. The process as defined by claim 75, wherein a first measure is carried out in an accumulation chamber at time t1 with a proportion of carrier fluid of c1, and then at least one second measure is carried out in the accumulation chamber at time t2 with a proportion of carrier fluid of c2 being lower than c1.

85. The process as defined by claim 75, wherein, first in the flowing channel, a mixture of candidate compounds A and B is introduced with relative proportions of respectively a/(a+b) and b/(a+b), then a measure in an accumulation chamber is carried out for said proportions, then a second proportion of a′/(a′+b′) and b′/(a′+b′) is introduced, and then a measure is carried out in an accumulation chamber of proportions respectively of (a+a′)/(a+a′+b+b′) and (a+a′)/(a+a′+b+b′).

86. The process as defined by claim 75, wherein the device comprises at least two flowing channels, being parallel on at least one segment thereof.

87. The process as defined by claim 75, wherein the device has accumulation chambers associated with the flowing channels, and wherein the following is carried out:
first step:
in a first flowing channel, a mixture of candidate compounds A and B is introduced with relative proportion of respectively a/(a+b) and b/(a+b), then a measure is carried out in an accumulation chamber of said channel for said proportions,
in a second flowing channel, a mixture of candidate compounds A and B is introduced with relative proportion of respectively a′/(a′+b′) and b′/(a′+b′), then a measure in an accumulation chamber of said channel is carried out for said proportions,
optionally, then second step:
the measure is repeated with same proportions in respective accumulation chambers but with different concentrations of carrier fluid.

88. The process as defined by claim 75, wherein one liquid mixture is provided into one of the flowing channels and is a reference liquid mixture, having a known physical and/or chemical property.

89. The process as defined by claim 75, wherein:
at least two flowing channels each have a different length surrounded by evaporation chamber(s),
the same liquid mixture is introduced in at least the two channels,
the concentration in carrier fluid is different in the different channels, and
a solid and/or concentrated phase forms differently in the different channels.

90. The process as defined by claim 75, wherein different information about solubility and/or crystallization kinetics and/or crystal growth kinetics is generated.

91. The process as defined by claim 75, wherein:
two different liquid mixtures with an identical or different carrier fluid and one or several identical or different candidate chemical compound(s) are flowed into the flowing channels.

92. The process as defined by claim 75, wherein the different liquid mixtures are provided in combination with an associated microfluidic mixing device.

93. The process as defined by claim 75, wherein:
the carrier fluid is identical in each channel,
the candidate chemical compound(s) are different in each channel and/or are mixtures of identical compounds with different concentrations in each channel, and
the process provides an array of:
different concentrations of carrier fluid and or residence times along the channels, with
different compositions of matter through parallel segments of the channels.

94. The process as defined by claim 75, wherein:
the carrier fluid is different in each channel,
the candidate chemical compound(s) is/are identical in each channel and/or is a mixture of an identical compound with another compound, optionally with different concentrations, and
the process provides an array of:
different concentrations of carrier fluid and or residence times along the channels, with
different compositions of matter through parallel segments of the channels.

95. The process as defined by claim 94, comprising conducting measurements or observations for all or a part of the compositions of matter of the array.

96. The process as defined by claim 75, wherein measurements are performed by spectroscopy means, conductimetry, rheology or optical observation, or image analysis.

97. The process as defined by claim 96, wherein the measurements are computed to provide:
phase diagrams,
crystallization diagrams,
crystallization kinetics data,
crystal growth kinetics data,
solubility data,
nucleation of solids data,
kinetics of chemical reactions,
formulation data, and/or
material engineering data.

98. The process as defined by claim 75, wherein the candidate chemical compound(s) comprise(s):
biological molecules, biological polymers,
non-biological molecules, synthetic polymers, surfactants, inorganic particles.

99. The process as defined by claim 75, wherein:
the carrier fluid is a solvent of at least some of candidate chemical compounds, the mixture of the carrier fluid and the candidate being in a form of:
a solution,
an emulsion of liquid droplets, or
a dispersion of solid particles.

100. The microfluidic device as defined by claim 51, the evaporating chamber or zone surrounding the width and/or depth of the channel, along at least ½ of the length of the flowing channel.

101. The microfluidic device as defined by claim 51, the evaporating chamber or zone surrounding the width and/or depth of the channel, along at least ⅔ of the length of the flowing channel.

102. The microfluidic device as defined by claim 51, the evaporating chamber or zone surrounding the width and/or depth of the channel, along at least ⅓ of the length and along at least 100 times the width and/or depth of the flowing channel.
